(19) United States
(12) Patent Application Publication
(10) Pub. No.: US 2010/0256125 A1
(43) Pub. Date: Oct. 7, 2010

(54) USE OF IMPROVED TOLUIDINE BLUE IN PHOTODYNAMIC THERAPY

(75) Inventors: Mark BRIDE, Peoria, AZ (US); Hal SIEGEL, Scottsdale, AZ (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: ZILA PHARMACEUTICALS, INC., Scottsdale, AZ (US)

(21) Appl. No.: 12/419,252

(22) Filed: Apr. 6, 2009

(51) Int. Cl. A61K 31/54

(52) U.S. Cl. 514/226.2

(57) ABSTRACT

A method of treating oral cancer is provided. The method comprises the steps of providing at least one photodynamic treatment comprising the steps of (i) applying a composition comprising improved toluidine blue to oral cells; wherein the composition comprises a greater percentage by weight of the conformational isomers of toluidine blue compared to the N-demethylated derivatives thereof; and wherein the composition localizes to oral cancer cells; and activating the applied composition, thereby killing the oral cancer cells.

### FIELD OF THE INVENTION

The present invention relates to photodynamic therapy, and more particularly, to use of particular compositions of toluidine blue in photodynamic therapy.

### BACKGROUND OF THE INVENTION

Oral cancer has one of the lowest survival rates of all cancers with little change in overall survival in the past several decades. If detected early, the prognosis for oral cancer is excellent; however, more than ⅔ of oral cancers are detected in advanced stages of disease. Detected at a late stage, the prognosis is poor, morbidity of treatment and economic cost of care is high. Once detected, treatment is inefficient.

Current methods of treatment include photodynamic therapy using one or more photosensitizers. However, current therapy is inefficient, time-consuming, and not very effective.

### BRIEF SUMMARY OF THE PREFERRED EMBODIMENTS

According to one aspect of the present invention, a method of treating oral cancer is provided. The method includes the steps of providing at least one photodynamic treatment comprising the steps of (i) applying a composition comprising an improved toluidine blue “ITB” to oral cells; wherein the composition comprises a greater percentage by weight of the conformational isomers of toluidine blue compared to the N-demethylated derivatives thereof; and wherein the composition localizes to oral cancer cells. The method further comprises the step of directing a source of coherent light to the applied composition, thereby killing the oral cancer cells. Preferably, lesion reduction is higher using ITB than using other compositions of toluidine blue “STB.” Preferably, the extent of intravascular thrombosis is greater using ITB compared to using STB.

In preferred embodiment, the composition comprises conformational isomers of toluidine blue having the ring methyl in the −2 position and conformational isomers of toluidine blue having the ring methyl in the −4 position; wherein the conformational isomer having the ring methyl in the −2 position comprises at least about 58% by weight of the total organic dye content of the composition. In another preferred embodiment, the composition comprises a first group of components, comprising (i) the conformational isomers of toluidine blue having the ring methyl in the −2 position; N-demethylated derivatives thereof having the ring methyl in the −2 position; and N,N-demethylated derivatives thereof having the ring methyl in the −2 position; and a second group of components comprising (ii) the conformational isomers of toluidine blue having the ring methyl in the −4 position; N-demethylated derivatives thereof having the ring methyl in the −4 position; and N,N-demethylated derivatives thereof having the ring methyl in the −4 position. The ratios of the combined areas of the 254 nm HPLC peaks representing the compounds in (i) to the combined areas of the peaks representing the compounds in (ii) being at least about 2.5:1.

In another preferred embodiment, the composition comprises (a) conformational isomers of toluidine blue, having the ring methyl in the −2 position; conformational isomers of toluidine blue, having the ring methyl in the −4 position; and (b) N-demethylation derivatives of the conformational isomers of toluidine blue having the ring methyl in the −2 position; N-demethylation derivatives of the conformational isomers of toluidine blue having the ring methyl in the −4 position. Preferably, the ratio of the combined areas of the 254 nm HPLC peaks representing the isomers to the combined areas of the peaks representing the N-demethylation derivatives being at least about 6:1.

According to another aspect of the present invention, a kit is provided. The kit preferably comprises a composition comprising ITB; wherein the composition comprises a greater percentage by weight of the conformational isomers of toluidine blue compared to the N-demethylated derivatives thereof. The kit also includes instructions for using the composition for killing oral cancer cells; wherein the instructions include the steps of (i) applying the composition to oral cells; wherein the composition localizes to oral cancer cells; and (ii) directing a source of coherent light to the applied composition; thereby killing oral cancer cells.

Other objects, features and advantages will become apparent to those skilled in the art from the following detailed description. It is to be understood, however, that the detailed description and specific examples, while indicating exemplary embodiments, are given by way of illustration and not limitation. Many changes and modifications within the scope of the following description can be made without departing from the spirit thereof, and the description should be understood to include all such variations.

### BRIEF DESCRIPTION OF THE DRAWINGS

Like numerals refer to like parts throughout the several views of the drawings.

FIG. 1 is a HPLC diagram of the composition of standard toluidine blue; wherein the areas of peaks 5 through 8 are such that, for example, peak 5<peak 7<peak 6<peak 8;

FIG. 2 is a HPLC diagram of the composition of the toluidine blue preferably used in the present invention; wherein the areas of peaks 5 through 8 are such that, for example, peak 5<peak 6<peak 7<peak 8;

FIG. 3 is a diagram of a preferred process of producing toluidine blue that is used in the present invention;

FIG. 4 is a diagram of the effects of the use of (3) light intensities on vascular and cellular photodynamic effects; and

FIG. 5 is a chart of the total combined vascular and cellular score from data pooled from each PDT parameter.

### DETAILED DESCRIPTION OF THE PREFERRED EMBODIMENTS

The present invention relates to the use of toluidine blue, preferably a particular compositions of toluidine blue as discussed herein, in photodynamic therapy.

Photodynamic therapy (“PDT”) is a technique for the treatment of various abnormalities or disorders of the skin or other epithelial organs or mucosa, especially cancers or precancerous lesions, as well as non-malignant lesions. Photodynamic therapy involves the application of photosensitizing agents to the affected area of the body, followed by exposure to photoactivating light in order to activate the photosynthesizing agents and convert them into cytotoxic form, whereby the affected cells are killed or their proliferative potential diminished.

Specifically, the present invention relates to the use of toluidine blue as a photosensitizer having a composition that is in accordance with the compositions as disclosed in U.S. Pat. No. 6,086,852, the contents of which are herein incorporated by reference in its entirety. The toluidine blue as disclosed in U.S. Pat. No. 6,086,852 includes a greater percentage by weight of the conformational isomers of toluidine blue compared to the N-demethylation derivatives, or the N,N-demethylation derivatives thereof.

In a preferred embodiment, the toluidine blue prepared in accordance with U.S. Pat. No. 6,086,852 is available from Zila, Inc. (or its subsidiaries) in Scottsdale, Ariz., and is commercially available under the name “ZTC,” Zila Tolonium Chloride,” and/or “OraTest.” For the sake of simplicity, the toluidine blue as prepared in accordance with U.S. Pat. No. 6,086,852 is referred to herein as “ITB” (or “improved toluidine blue”). In other embodiments, the ITB may be available from other entities besides Zila, Inc.

“Standard toluidine blue” or “standard toluidine blue O” refers to toluidine blue, for example, that includes a lower percentage by weight of the conformational isomers of toluidine blue compared to the N-demethylation derivatives, or the N,N-demethylation derivatives thereof (as compared to the percentage by weight of the conformational isomers of toluidine blue compared to the N-demethylation derivatives, or the N,N-demethylation derivatives thereof in ITB). For sake of simplicity, standard toluidine blue will be referred to herein as “STB.”

In other embodiments, the use of ITB in the present invention is not limited to the toluidine blue that is disclosed in U.S. Pat. No. 6,086,852 and may include any toluidine blue that comprises a greater percentage by weight of the conformational isomers of toluidine blue; and/or greater combined areas the HPLC of peak 8 and/or peaks 8 and 7 as disclosed in U.S. Pat. No. 6,086,852.

In a preferred embodiment, the ITB includes from about 20 to about 90 percent by weight of peaks 5 through 8; more preferably, the ITB includes from about 30 to about 80 percent by weight of peaks 5 through 8; and most preferably from about 40 to about 75 percent by weight of peaks 5 through 8. For example, the ITB may include from about 48% to about 73% by weight of peaks 5 through 8. The amount (i.e., the weight percentage) of peaks 5-8 in the total composition of ITB may be referred to as the “organic component of ITB.”

In a preferred embodiment, the organic component of the ITB includes from about 0.5 to about 15% by weight of peak 5; more preferably from about 1 to about 10% by weight of peak 6; most preferably from about 2 to about 5 % by weight of peak 5.

In a preferred embodiment, the organic component of the ITB includes from about 1 to about 20 percent by weight of peak 6; more preferably from about 2 to about 10 percent by weight of peak 6; most preferably from about 4 to about 8 percent by weight of peak 6.

In a preferred embodiment, the organic component of the ITB includes from about 5 to about 40 percent by weight of peak 7; more preferably from about 10 to about 30 percent by weight of peak 7; and most preferably from about 14 to about 24 percent by weight of peak 7.

In a preferred embodiment, the organic component of ITB includes from about 10 to about 60 percent by weight of peak 8; more preferably from about 15 to about 45 percent by weight of peak 8; most preferably from about 25 to about 37 by weight of peak 8.

In other embodiments, the percent by weight of peaks 5 through 8 may be greater or less than the foregoing.

In other embodiments, ITB may be combined with any other photosensitizers and used as a combination.

Use of ITB in photodynamic therapy (and as a photosensitizer) results in a surprisingly more efficient cancer regimen than use of STB, or any other agent or photodynamic agent or a combination thereof. For example, ITB in photodynamic therapy results in a surprisingly greater lesion reduction than STB in photodynamic therapy does. As used herein, “lesion reduction” may refer to one or more of the following: lesion regression, reduction in lesion size and/or density; reduction in cell proliferation, increased or induced cellular necrosis; increase in apoptosis, reduction in angiogenesis, activation of tumor suppressor genes/proteins, reduction in cancer or precancer, increase in intravascular thrombosis and the like.

Lesion reduction may be measured by any means known in the art. For example, lesion reduction may be measured by measuring size, density, volume, histological analysis, or the like. Additionally, lesion reduction may be determined by molecular means, i.e., by measuring an increase in the proteins encoded by tumor suppressor genes, i.e., an increase in translation (or an increase in transcription, etc. as is known in the art), measuring a reduction in proteins encoded by proto-oncogenes, by measuring DNA methylation, or the like, using standard molecular techniques. Samples treated with STB may be compared with samples treated with ITB, and the like. Lesion reduction may also be measured by comparing the rate of angiogenesis. Lesion reduction in samples treated with ITB may be compared with samples treated with STB.

In a preferred embodiment, as used herein, activated ITB is used to kill cancer cells. As used herein when coherent light is directed to ITB or STB, it becomes “activated.”

As used herein, “cancer” or “cancerous” cells may refer to cells and/or tissue that have undergone malignancy. In another embodiments, “cancer” or “cancerous” may refer to, for example, any abnormal cells/tissue, such as abnormal epithelial tissue (as used herein, tissue characterized as “abnormal and/or cancerous” may include any/all of the following: pre-cancerous tissue; cancerous tissue; tissue experiencing angiogenesis; tissue exhibiting molecular/genetic signs of precancer/cancer; tissue having cells with abnormal apoptotic pathways, etc.). Additionally, as used herein, “oral cancer” is not limited to oral cancer and may include cancer in other parts of the body.

As used herein, the phrase “killing [oral] cancer cells” and/or “wherein at least a majority of [oral cancer] cells are killed” may refer to the point where there is no clinical sign of cancer and/or the point where there is a reduction in malignancy/reduction in the lesion or decrease in the number of molecular markers associated with cancer cells. Additionally, “killing oral cancer cells” may also refer to reducing proliferation of cancer cells; activating the apoptotic pathway of cancer cells; reducing angiogenesis and/or vascularization, inhibiting the cell cycle pathway, or the like.

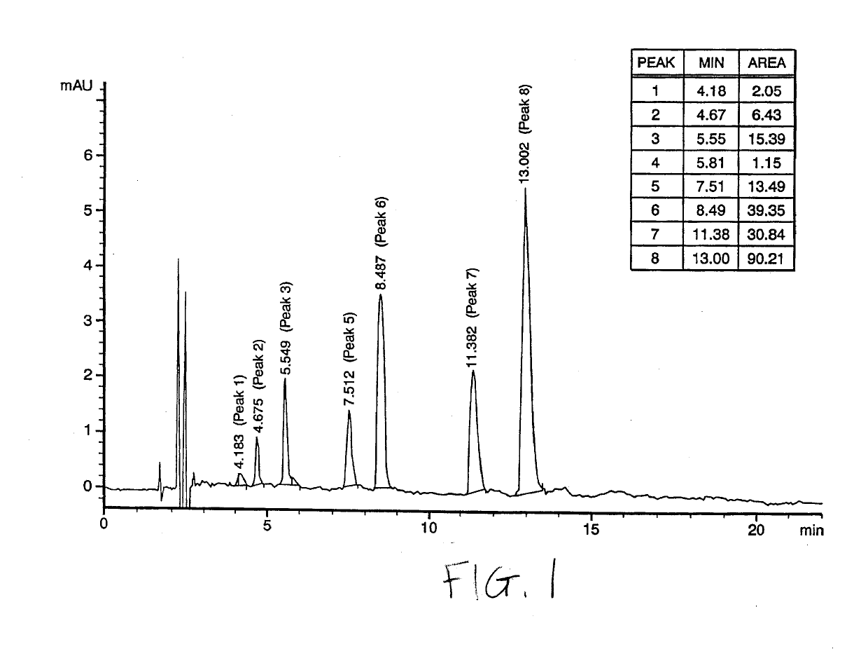

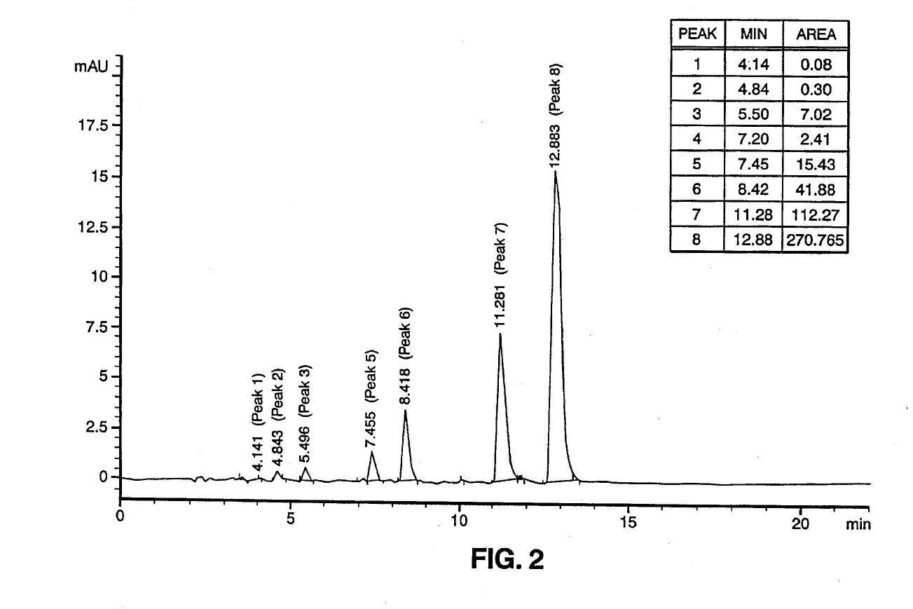

For example, the composition of STB is different from the ITB as used in the present invention. More specifically, referring to FIG. 1, which depicts the 254 nm HPLC for STB, the 254 nm peaks 7 and 8 refer to the conformational isomers of toluidine blue and the 254 nm peaks 5 and 6 refer to N-demethylated derivatives of the conformational isomers of toluidine blue. The relative quantities of the conformational isomers of toluidine blue O to their N-demethylated derivatives is less than 4:1 in FIG. 1. However, referring to FIG. 2, which depicts a 254 nm HPLC analysis of the ITB as used in the present invention, the peak ratio of ITB conformational isomers to N-demethylation derivatives thereof is preferably greater than about 6:1. In a preferred embodiment, peak 8 is at least about 58% of the total organic dye content of the ITB. The HPLC peaks 7 and 8 are over 6 times greater than the combined areas of peaks 5 and 6. For example, the overall purity of the ITB is preferably at least greater than about 50-75%, compared to 2-10% for most prior art compositions or STB.

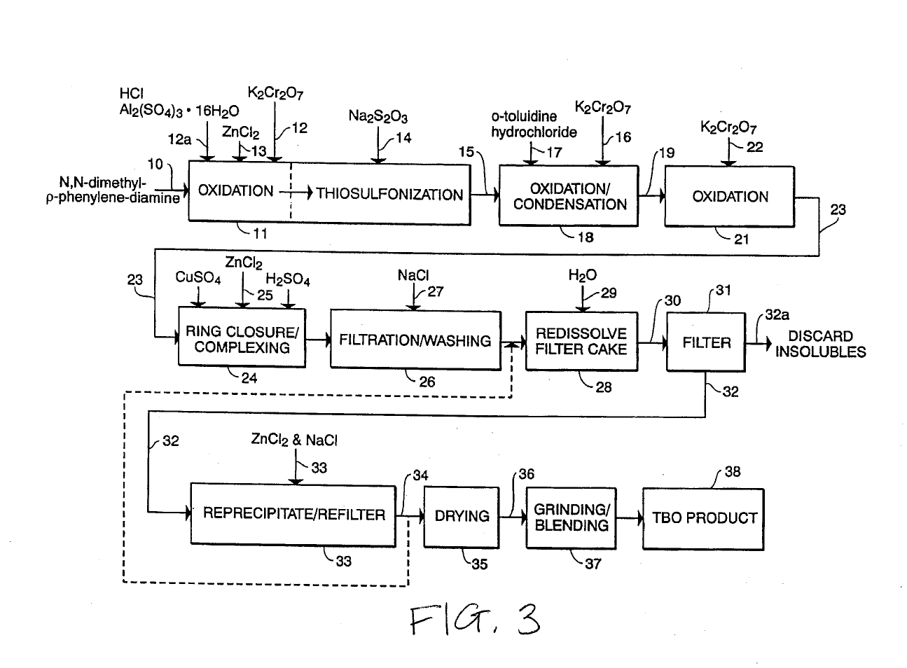

In a preferred embodiments and referring to FIG. 3, ITB may be prepared according to the following methods, described below and as disclosed in U.S. Pat. No. 6,086,852. However, it is to be understood that the preparation of the ITB for use as a photosensitizer is not limited to the following methods. Any method that produces a higher amount of peak 8, or peaks 8 and 7, may be used.

### Preferred Methods of Preparation of Improved Toluidine Blue (“ITB”)

In a preferred embodiment, a first reaction mixture having a first intermediate is produced after oxidation and thiosulfonization.

### Formation of the First Reaction Mixture

### Oxidation

An aqueous solution of N,N-dimethyl-p-phenylene diamine 10 is oxidized at less than 10° C., using a suitable oxidizing agent, such as potassium dichromate 12 in the presence of acid, aluminum sulfate and a reagent 13. Any suitable oxidizing agent may be used. The reagent may be a complexing agent.

Thiosulfonization

A source of thiosulfate ions 14, e.g., sodium thiosulfate, is added to form a first reaction mixture 15 containing the first intermediate, 2-Amino-5-dimethylaminophenyl thiosulfuric acid.

### Formation of Second Reaction Mixture

Oxidation/Condensation

The first reaction mixture 15 is then further reacted, preferably at a temperature of not greater than about 10° C., with an oxidizing agent 16, i.e., potassium dichromate, and o-toluidine hydrochloride 17, in a condensation step 18 to form the second intermediate, a condensation product, indamine thiosulfuric acid, in the second reaction mixture 19.

### Formation of Third Reaction Mixture

Oxidation

The second reaction mixture 19 is then further oxidized 21, preferably by addition of a suitable oxidizing agent 22, i.e., potassium dichromate, preferably at a temperature of no more than about 10° C. It is to be understood that any suitable oxidizing agent may be used.

Ring Closure/Complexing

This is followed by the addition of copper sulfate, complexing agent 25, e.g., zinc chloride, and acid. The mixture is heated to 100° C. to effect closure of the indamine ring, forming TBO in a third reaction mixture 24. At this point the TBO is separated from the third reaction mixture and purified.

### Separation/Purification of TBO

Filtration/Washing

In a preferred embodiment, the TBO is precipitated from the third reaction mixture 24 by complexation of 24 with a suitable complexing agent 25, e.g., zinc chloride, to form the complex TBO-zinc chloride double salt. The precipitate is filtered 26 from the liquid phase and washed with sodium chloride solution 27. The washed filter cake is then redissolved 28 in a volume of water 29 to form a TBO solution 30, which is then filtered 31 to remove undissolved solids 32a, which are discarded. Zinc Chloride, followed by a concentration of sodium chloride 33 is then added to the filtrate 32 to again precipitate the TBO-zinc chloride/TBO hydrochloride filter cake 34.

As indicated by the dashed line 35, the TBO filter cake 34 can be redissolved, filtered, re-precipitated and reisolated multiple times to achieve the desired degree of purity and yield of TBO. The final purified filter cake complex product 34 is then dried 35, e.g., in conventional convection oven and/or vacuum oven and the dried filter cake 36 is ground and blended 37 to yield the final TBO product 38. The final TBO product contains both the zinc chloride double-salt of TBO and the chloride salt of TBO.

In a preferred embodiment, the ITB used as a photosensitisizer in the present invention be comprised of:

(a) the conformational isomers of toluidine blue, the compounds having the structures

(referred to herein as peak 8 or conformational isomer of toluidine blue having the ring methyl in the −2 position)

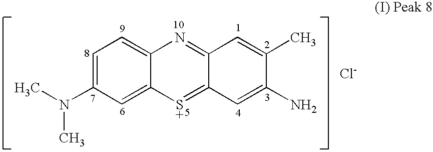

and

(referred to herein as peak 7 or conformational isomer of toluidine blue having the ring methyl in the −4 position)

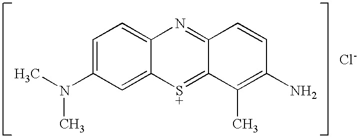

(b) the N-demethylation derivatives of said isomers, the compounds having the structures

(referred to herein as peak 6 or the N-demethylated derivative of the conformational isomer of toluidine blue having the ring methyl in the −2 position)

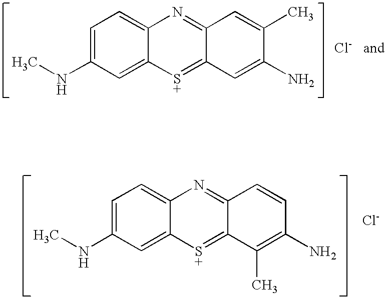

(referred to herein as peak 5 or the N-demethylated derivative of the conformational isomer of toluidine blue having the ring methyl in the −4 position)

the ratio of the combined areas of the 254 nm HPLC peaks representing the isomers to the combined areas of the peaks representing the N-demethylation derivatives being at least about 6:1.

In a preferred embodiment, ITB may be comprised of:

(a) a first group of components, comprising a conformational isomer of toluidine blue having the ring methyl group in the −2 position, the compound having the structure

(referred to herein as peak 8)

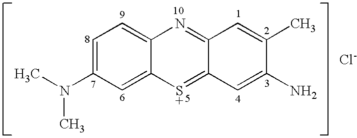

a N-demethylation derivative thereof, the compound having the structure

(referred to herein as peak 6)

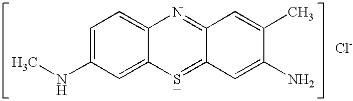

an N,N-demethylation derivative thereof, the compound having the structure

(referred to herein as peak 3)

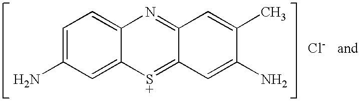

(b) a second group of components, comprising a conformational isomer of toluidine blue having the ring methyl group in the −4 position, the compound having the structure

(referred to herein as peak 7)

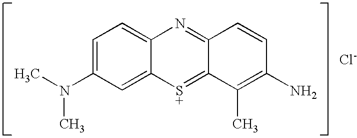

an N-demethylation derivative thereof, the compound having the structure

(referred to herein as peak 5 or the N-demethylation derivative of the conformational isomer of toluidine blue having the ring methyl in the −4 position)

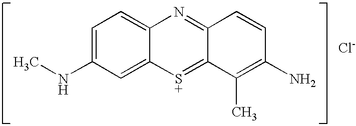

an N,N-demethylation derivative thereof, the compound having the structure

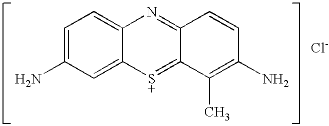

(referred to herein as peak 3 or the N,N-demethylation derivative of the conformational isomer of toluidine blue having the ring methyl in the −4 position)
the ratio of the combined areas of the 254 nm HPLC peaks representing the first group to the combined areas of the 254 nm HPLC peaks representing the second group being at least about 2.5:1.

In a preferred embodiment, the ITB may be comprised of the conformational isomers of toluidine blue, namely the compounds having the structures

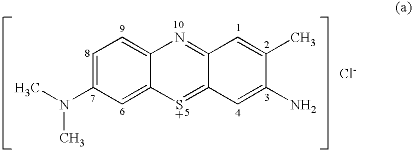

[peak 8 or the conformational isomer of toluidine blue having the ring methyl in the −2 position] and

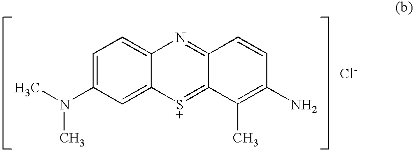

[peak 7 or the conformational isomer of toluidine blue having the ring methyl in the −4 position]

in which the isomer in (a) comprises at least about 58% by weight of the total organic dye content of the composition.

In a preferred embodiment, the ITB as prepared in the foregoing is applied and activated in one or more treatments or photodynamic treatments in order to kill oral cancer cells.

It is to be noted that “photodynamic treatment” as used herein, comprises at least one application of ITB and at least one activation of the applied ITB. Accordingly, “photodynamic treatment” as used herein may refer to applying the ITB any number of times and for any duration, or the like. Likewise, “photodynamic treatment” as used herein may refer to activating the applied ITB any number of times, and for any duration and/or intensity. This includes activating the ITB with coherent light, for example, of varying duration and/or intensity in one treatment.

Each photodynamic treatment preferably comprises the steps of applying a composition comprising ITB to oral cells and subsequently activating the composition and/or the ITB. As indicated in the foregoing, the use of ITB with a greater percentage by weight of peaks 8 and/or peaks 7 and 8 results in a cancer treatment regimen that is much more efficient.

In a preferred embodiment, the ITB comprises equal to or greater than about 5 percentage by weight of peak 8 (of the total composition of ITB); more preferably equal to or greater than about 10 percentage by weight of peak 8; most preferably equal to or greater than about 15 percentage by weight of peak 8.

In a preferred embodiment, the ITB comprises equal to or greater than about 10 percentage by weight of peaks 8 and 7 (of the total composition of ITB); more preferably equal to or greater than about 30 percentage by weight of peaks 8 and 7; most preferably greater than or equal to about 40 percentage by weight of peaks 8 and 7.

It is to be understood that the ITB may be applied alone or as a part of a composition.

In a preferred embodiment, the step of applying the composition comprises the steps of applying the composition from about 0.1 to about 10 minutes. In a more preferred embodiment, the step of applying the composition comprises the steps of applying the composition from about 0.3 to about 5 minutes. In a highly preferred embodiment, the step of applying the composition comprises the steps of applying the composition from about 0.5 to about 2.5 minutes. Preferably, the composition is applied for about 1 minute. In other embodiments, the composition may be applied for any other suitable duration. Preferably, the composition is rinsed prior to irradiation. The composition may be rinsed with acetic acid, for example, or any other suitable chemical, or water. In other embodiments, the composition is rinsed during, or after, irradiation.

In a preferred embodiment, the step of activating the composition comprises the step of directing a source of coherent light to the applied composition. Preferably, the source of coherent light is laser light. In other embodiments, any other source of coherent light may be used. In yet other embodiments, any other means of photoactivating ITB may be used without departing from the scope of the present invention.

In a preferred embodiment, the step of directing the source of coherent light to the applied composition comprises exposing the applied composition to the source of coherent light for a specified duration. In a preferred embodiment, the duration of exposure is from about 0.1 to about 60 minutes. In a more preferred embodiment, the duration of exposure is from about 3 to about 30 minutes. In a highly preferred embodiment, the duration of exposure is from about 5 to about 10 minutes. Preferably, the exposure duration is selected from the group consisting of 6, 9, and 12 minutes. In other embodiments, any the composition may be activated for any suitable duration without departing from the scope of the present invention.

In a preferred embodiment, the intensity of the source of coherent light is from about 10 J/cm2 to about 100 J/cm2; more preferably from about 20 J/cm2 to about 90 J/cm2; and most preferably from about 30 J/cm2 to about 60 J/cm2. In a highly preferred embodiment, the intensity of the source of coherent light is selected from the group consisting of 30 J/cm2; 45 J/cm2; 60 J/cm2. In other embodiments, the intensity may be any other numerical values, or a combination thereof, without departing from the scope of the present invention.

In a preferred embodiment, the laser light has a wavelength of from about 500 nm to about 800 nm; in a more preferred embodiment the laser light has a wavelength of from about 550 nm to about 750 nm; in a highly preferred embodiment, the laser light has a wavelength of from about 600 nm to about 650 nm. Most preferably, the laser light has a wavelength of about 633 nm. In other embodiments, the light may have any other wavelength without departing from the scope of the present invention.

In a preferred embodiment, from about 1 to about 6 photodynamic treatments are needed to kill the oral cancer cells that have ITB applied thereon; in a more preferred embodiment, from about 1 to about 4 photodynamic treatments are needed to kill the oral cancer cells that have ITB applied thereon; and in a highly preferred embodiment, from about 1 to about 2 photodynamic treatments are needed to kill the oral cancer cells that have ITB applied thereon. The number of treatments needed to kill the oral cancer cells using the methods of the present invention is surprisingly less than conventional methods, due to the higher percentage by weight of peak 8 or peaks 7 and 8 in the ITB and the resultant composition.

In a preferred embodiment, the ITB localizes to oral cancer cells. Preferably, at least a portion of the applied ITB remains unbound to any subcellular organelle. Preferably, at least a portion of the applied ITB localizes to the interstitial spaces of cancerous cells and/or stains the margins of the visually defined lesion.

The following examples are presented to enable those skilled in the art to understand and practice the invention and to identify the presently preferred embodiments thereof. These examples are provided for illustrative purposes and not to indicate the scope of the invention which is defined only by the appended claims.

### EXAMPLES OF THE PRESENT INVENTION

The following are examples of the methods/compositions for use in the present invention.

### Example 1

A routine visual examination of the oral cavity is made, noting the presence or absence of any lesions on the attached gingival, the buccal mucosa, the floor of the mouth, the hard and soft palate, and the dorsal, lateral, and the ventral tongue. The presence or absence of any lesions noted by this routine examination are recorded. Additionally, the presence or absence of clusters of blood vessels (i.e., angiogenesis) which may indicate new growth such as cancer is noted. Additionally, abnormal epithelial tissue may be detected, for example, using the chemiluminescent light source as described in U.S. Pat. No. 5,329,938 to Lonky and U.S. Patent Application Publication Nos. 2008/0255462 and 2006/0241494 to Bride, the entire contents of which are herein incorporated by reference in their entireties. The light source described in the patent is commercially available under VIZILITE. Further assessment of the noted sites is made, for example by tissue biopsy for standard histology or by molecular analysis, to determine whether the tissue is cancerous or harbors mutations which are in the pathway for the eventual development of invasive cancer. Molecular analysis may include PCR, such as microsatellite analysis, or the like.

### Example 2

After completing the routine examination in Example 1, the patient is then instructed to rinse the mouth with a 1% acetic acid solution for up to one minute and then expectorate. This is followed by a 1 minute application of 0.5% ITB, available from Zila, Inc., followed by a post-dye 1% acetic acid rinse. The ITB remaining after the acetic acid rinse preferably localizes to the cancerous cells. For example, the ITB staining preferably corresponds with the margins of the visually defined lesion. The ITB may be applied as described in U.S. Pat. No. 4,321,251 to Mashberg, the contents of which are herein incorporated by reference in its entirety.

### Example 3

After completing the procedure as set forth in Example 2, the applied ITB that localized to the cancerous cells is activated. Preferably, the applied ITB undergoes irradiation using laser light at 633 nm and at intensity levels of 30 J/cm2, 45 J/cm2, and 60 J/cm2. The applied toluidine blue is exposed at durations selected from the group of 6, 9, and 12 min.

### FURTHER EXAMPLES OF THE METHODS OF THE PRESENT INVENTION

In order to induce precancer and/or cancer and/or carcinogenesis, 45 Golden Syrian Hamsters were treated with 0.5% dimethyl-benzanthracene (DMBA) dissolved in mineral oil applied to their cheek pouches three times a week for 5, 7, or 12 weeks. 15 animals were included in each group. Lesion progression was visualized using non-invasive in vivo Optical Coherence Tomography (“OCT”) techniques for monitoring lesion progression.

A fiberoptic 3-D OCT/ODT probe was used, which permitted tomographic imaging of areas of up to 1.6 cm in length within approximately 1 minute. This probe was inserted into 2-D pullouts were optically generated from regions of interest, and scored according to a previously developed and validated OCT scale for oral dysplasia and malignancy.

At each of the foregoing time intervals, the ITB was applied in vivo in the following manner: A 60 second topical application of 1% acetic acid was followed by a 1 minute application of 0.5% TBO and a post-dye 1% acetic acid rinse. Cheek pouches underwent irradiation using laser light at 633nm at previously identified parameters: 30 J/cm², 45 J/cm², 60 J/cm² at exposure durations of 6, 9, and 12 min. 24 hours after photodynamic therapy, animals were sacrificed and cheek pouch tissues underwent routine histopathological preparation and evaluation. The following semi-quantitative scale was used for evaluating vascular and cellular PDT effects:

| A: Vascular | B: Cellular |
|---|---|
| 0 - no change | 1 - mile-moderate |
| 1 - mild-moderate intravascular thrombosis | cellular thermal damage |
| 2 - moderate-sever intravasucal thrombosis | 2 - moderate-severe |
| | cellular thermal damage |

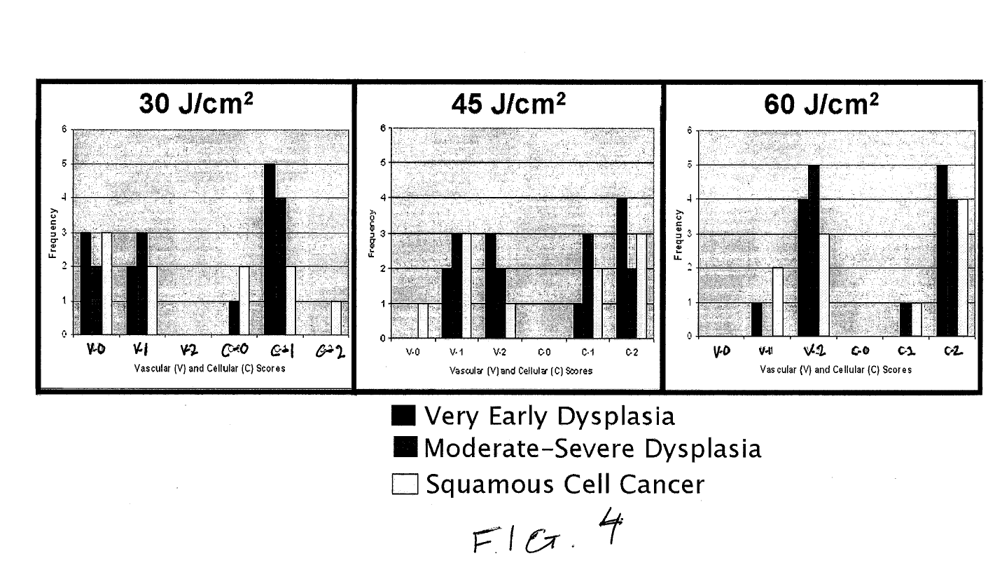

Referring now to FIG. 4, the vascular and cellular scores at various light intensities and stages of dysplasia/cancer can be seen. The reference to “V-0, V-1, V-2, C-0, C-1, and C-2” refers to the vascular or cellular score. As indicated in the table above, the greater the vascular or cellular score, the greater the PDT photodestruction.

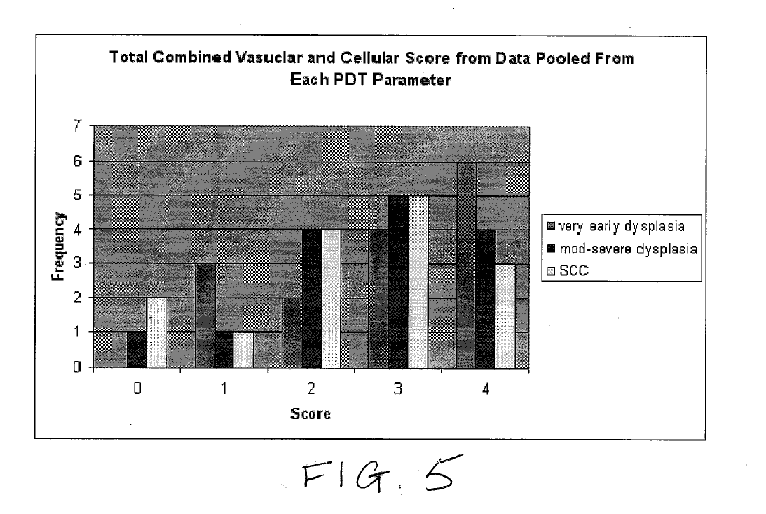

Referring to FIG. 5, the total combined vascular and cellular scores from data pooled from each PDT parameter (or light intensity). It is noted that the higher the total score, the more effective the PDT therapy.

Evidence of a positive ITB stain was observed on all cheek pouches that underwent 6 weeks of carcinogenesis and dye application, and one control cheek pouch with a negative staining result.

ITB mini-granules were visible below the tissue surface in samples where the keratinized layer had been comprised or in areas with concentrated cell damage and death. Further, scattering of the granules was visible in the epithelium just below the keratinized layer. This was typically seen in lesions with mild to moderate dysplasia, where thinning of discontinuity of the surface keratinized layer was observed.

In areas of cellular damage and death, zones of ITB granules were visible within tumor “whorls” where there were dead and dying cells. In areas of what happened to be high cell turnover, often with neovascularization and without may dead and dying cells, ITB presence was much reduced.

Additionally, positive ITB staining appeared to correspond with the margins of the visually defined lesion. The staining was visually unchanged after fixation in 2% paraformaldehyde. After the tissues were processed, however, the ITB bled out, and moved in the intercellular spaces. Accordingly, at least a portion of the ITB remained unbound and likely affected by a diffusion gradient.

The embodiments of the present invention described above are merely illustrative and are not to be construed as exhaustive. Those skilled in the art may make numerous uses of, and departures from, such embodiments without departing from the spirit and the scope of the present invention. Accordingly, the scope of the present invention is not to be limited to or defined by such embodiments in any way, but rather, is defined solely by the following claims.

What is claimed is:

1. A method of treating oral cancer comprising:
(a) providing at least one photodynamic treatment comprising the step of:
(i) applying a composition to oral cells; wherein the composition comprises a greater percentage by weight of the conformational isomers of toluidine blue compared to the N-demethylated derivatives thereof; and wherein the composition localizes to oral cancer cells; and
(b) directing a source of coherent light to the applied composition, thereby killing the oral cancer cells.

2. The method of claim 1, wherein the composition comprises:
(a) conformational isomers of toluidine blue having the ring methyl in the −2 position and
(b) conformational isomers of toluidine blue having the ring methyl in the −4 position
wherein (a) comprises at least about 58% of the total organic dye content of the composition, and wherein the composition localizes to oral cancer cells.

3. The method of claim 1, wherein the lesion reduction is higher using the composition of claim 1 than using other toluidine blue compositions.

4. The method of claim 1, wherein the composition comprises:
(a) a first group of components, comprising:
conformational isomers of toluidine blue having the ring methyl in the −2 position; and
N-demethylated derivatives of toluidine blue having the ring methyl in the −2 position; and
N,N-demethylated derivatives of toluidine blue having the ring methyl in the −2 position; and
(b) a second group of components, comprising:
conformational isomers of toluidine blue having the ring methyl in the −4 position;
N-demethylated derivatives of toluidine blue having the ring methyl in the −4 position; and
N,N-demethylated derivatives of toluidine blue having the ring methyl in the −4 position;
the ratio of the combined areas of the 254 nm HPLC peaks representing the compounds in (a) to the combined areas of the peaks representing the compounds in (b) being at least about 2.5:1.

5. The method of claim 1, wherein the source of coherent light is laser light.

6. The method of claim 1, the extent of intravascular thrombosis is greater using the composition of claim 1 compared to other toluidine blue compositions.

7. The method of claim 3, wherein the lesion reduction is higher using the composition of claim 1 compared to other toluidine blue compositions.

8. The method of claim 1, wherein the composition comprises:
(a) conformational isomers of toluidine blue, having the ring methyl in the −2 position; and
conformational isomers of toluidine blue, having the ring methyl in the −4 position; and
(b) N-demethylation derivatives of the conformational isomer of toluidine blue having the ring methyl in the −2 position; and
N-demethylation derivatives of the conformational isomer of toluidine blue having the ring methyl in the −4 position;
the ratio of the combined areas of the 254 nm HPLC peaks representing the isomers to the combined areas of the peaks representing the N-demethylation derivatives being at least about 6:1.

9. A kit for killing oral cancer cells, comprising:
(a) the composition of claim 1;
(b) instructions for using the composition for killing oral cancer cells; wherein the instructions include the steps of
(i) applying the composition of claim 1 to oral cells; wherein the composition localizes to oral cancer cells; and
(ii) directing a source of coherent light to the applied composition; thereby killing oral cancer cells.

10. The kit of claim 9, wherein the source of coherent light is laser light.
